(19) United States
(12) Patent Application Publication
(10) Pub. No.: US 2021/0012880 A1
(43) Pub. Date: Jan. 14, 2021

(54) FOOD PRODUCT INFORMATION PROVIDING SYSTEM, DEVICE, METHOD, AND PROGRAM

(71) Applicants: NISSIN FOODS HOLDINGS CO., LTD., Osaka-shi, Osaka (JP); NISSIN FOOD PRODUCTS CO., LTD., Osaka-shi, Osaka (JP)

(72) Inventors: Noritaka ANDO, Osaka-shi, Osaka (JP); Takanori SAKURAGI, Osaka-shi, Osaka (JP); Futoshi NAKAMURA, Osaka-shi, Osaka (JP); Tetsuya KAWASE, Osaka-shi, Osaka (JP)

(21) Appl. No.: 16/978,105

(22) PCT Filed: Jun. 5, 2019
(86) PCT No.: PCT/JP2019/022303

§ 371 Date: Sep. 3, 2020

(30) Foreign Application Priority Data
Jun. 5, 2018 (JP) 2018-108125

(51) Int. Cl. G16H 20/60; G16H 50/30; G06Q 30/02

(52) U.S. Cl. CPC G16H 20/60; G06Q 30/0283; G06Q 30/0207; G16H 50/30

(57) ABSTRACT

Provided is a food product information providing system that creates indices for health risks based on genetic information such as the results of genetic testing, biological information such as the results of a medical checkup, and lifestyle information such as the results of a lifestyle questionnaire from a user, and then selects one or more functional materials according to each risk index and provides the user with a food product or recipe that includes the selected functional material(s) and matches the preference of the user.

### TECHNICAL FIELD

The present invention relates to a food product information providing system, device, method, and program.

### BACKGROUND ART

In recent years, there have been growing problems with increased medical expenses and nursing care expenses along with aging. To suppress medical expenses and ensure a comfortable life after retirement, it is an important challenge to extend one's “healthy life expectancy” so that one can live his/her daily life without being restricted for health reasons.

To extend one's “healthy life expectancy,” it is necessary to find a “presymptomatic disease” state between a healthy state and a disease state in an early stage and improve it. However, how to deal with such a “presymptomatic disease” state is up to one's efforts.

Conventionally, there have been known a system (Patent Literature 1) that classifies a plurality of users into groups based on the results of a questionnaire from the users, and presents to a target user information about a functional material that has been determined to be effective for another user belonging to the same group as the target user, and a system (Patent Literature 2) that identifies a nutrient suitable for one's health condition based on his/her blood glucose level measured with a biosensor, for example, and determines a menu accordingly.

In addition, as a method of presenting health risks of individual users to the users, using a health information display device (Patent Literature 3) is known that collects from each user genome information, biological information, and behavior information, which are the health information on the user, and analyzes the accumulated health information on the plurality of users, thereby deriving the relevance between a combination of the type of genome derived from the genome information and the type of lifestyle derived from the biological information and behavior information and a diseases having a possibility of being caused in the future.

### CITATION LIST

### Patent Literature

Patent Literature 1: Japanese Patent No. 6245487
Patent Literature 2: Japanese Patent Laid-Open No. 2006-323791
Patent Literature 3: Japanese Patent No. 6212784

### SUMMARY OF INVENTION

### Technical Problem

However, since each user usually has a plurality of health risks, it is required to select an optimal functional material(s) corresponding to the plurality of health risks, and provide a food product or recipe that includes the functional material(s) while respecting the preference of the user.

### Solution to Problem

The present invention has been made to solve the aforementioned problem. In an embodiment of the present invention, indices are created for health risks based on genetic information such as the results of genetic testing, biological information such as the results of a medical checkup, and lifestyle information such as the results of a lifestyle questionnaire from a user. Then, one or more functional materials are selected according to each risk index, and a food product or recipe is provided to the user that includes the selected functional material(s) and matches the preference of the user.

### Advantageous Effects of Invention

According to an embodiment of the present invention, a user can easily and effectively take in functional materials that are effective in reducing his/her health risks by eating a food, such as a processed food product that he/she purchases on a daily basis. It should be noted that the functional materials do not include the pharmaceuticals regulated by the Pharmaceutical Affairs

### BRIEF DESCRIPTION OF DRAWINGS

FIG. 1 is a diagram illustrating an overview of a food product information providing system of the present embodiment.

FIG. 2 is a block diagram illustrating the configuration of the food product information providing system of the present embodiment.

FIG. 3 is a diagram illustrating an overview of a process of the food product information providing system of the present embodiment.

FIG. 4 is a chart illustrating an overview of various types of information input to an information input unit in which FIG. 4(a) illustrates an overview of genetic information input to a genetic information input unit, FIG. 4(b) illustrates an overview of biological information input to a biological information input unit, and FIG. 4(c) illustrates an overview of lifestyle information input to a lifestyle information input unit.

FIG. 5 is a chart illustrating an example in which a risk calculation unit calculates a manifest risk index and a potential risk index for each health risk based on genetic information, biological information, and lifestyle information.

FIG. 6 is a chart illustrating an exemplary process of the risk calculation unit to calculate a risk index for each health risk based on the manifest risk index and the potential risk index.

FIG. 7 is a chart illustrating an exemplary functional material DB for a material selection unit to select a functional material(s) suitable for reducing a specific health risk.

FIG. 8 is a view illustrating an exemplary display pattern of food product information on a terminal device.

FIG. 9 is a flowchart illustrating the processing operation of the food product information providing system of the present embodiment.

### DESCRIPTION OF EMBODIMENTS

Hereinafter, embodiments for implementing a food product information providing device and method according to the present invention will be described in detail with reference to the drawings. It should be noted that the present invention is not limited to the following embodiments or examples.

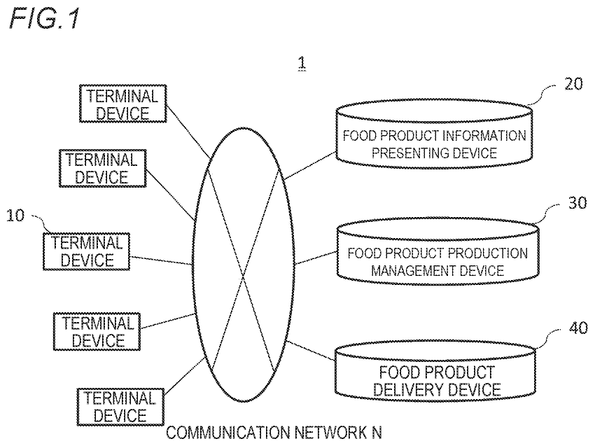

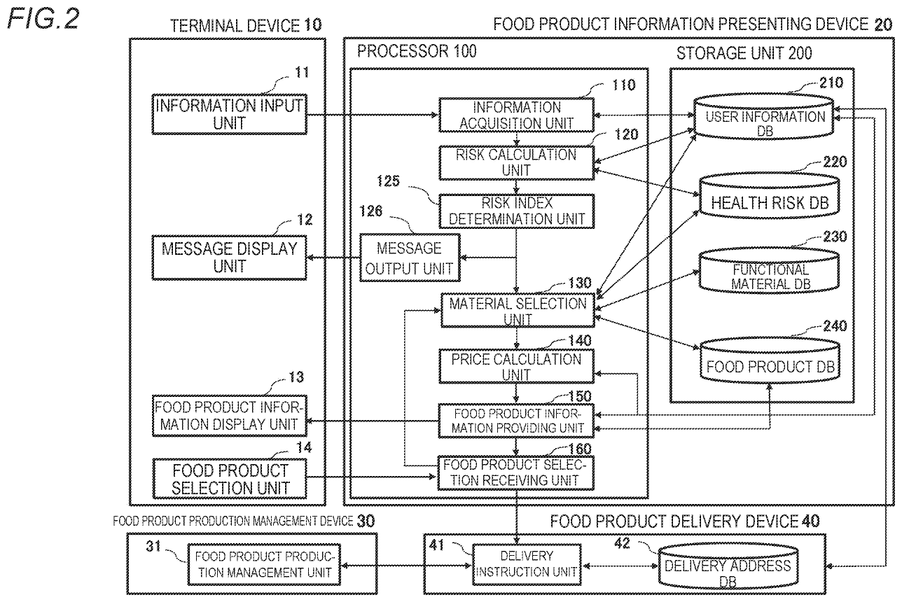

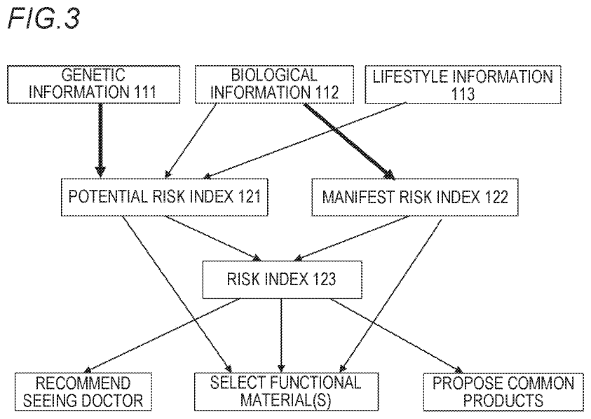

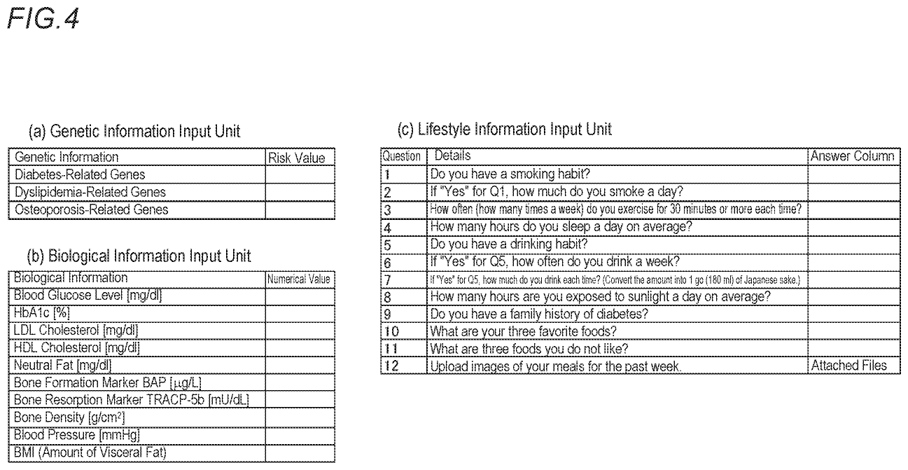

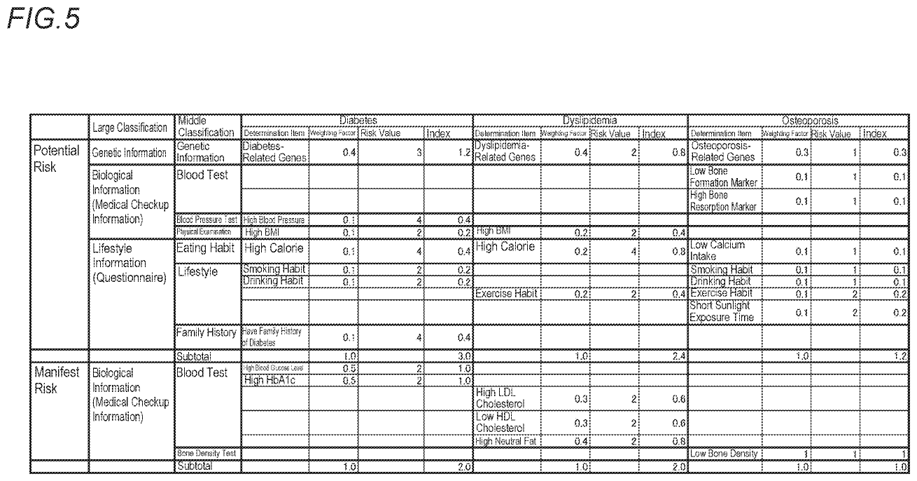

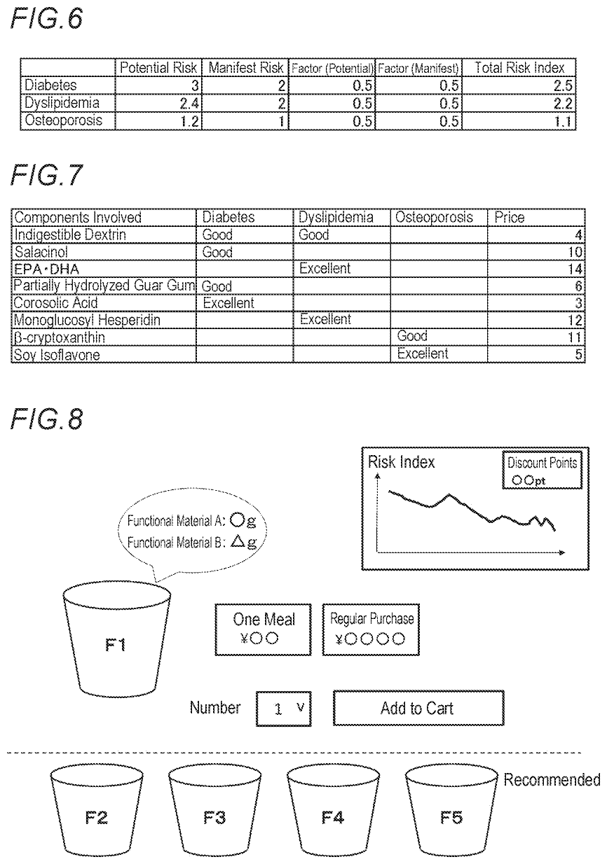

The configuration of a food product information providing system of the present embodiment will be described with reference to FIGS. 1 to 8. FIG. 1 is a diagram illustrating an overview of the food product information providing system of the present embodiment. FIG. 2 is a block diagram illustrating the configuration of the food product information providing system of the present embodiment. FIG. 3 is a diagram illustrating an overview of a process of the food product information providing system of the present embodiment. FIG. 4 is a chart illustrating an overview of various types of information input to an information input unit in which FIG. 4(a) illustrates an overview of genetic information input to a genetic information input unit, FIG. 4(b) illustrates an overview of biological information input to a biological information input unit, and FIG. 4(c) illustrates an overview of lifestyle information input to a lifestyle information input unit. FIG. 5 is a chart illustrating an exemplary process of a risk calculation unit to calculate a manifest risk index and a potential risk index for each health risk based on genetic information, biological information, and lifestyle information. FIG. 6 is a chart illustrating an exemplary process of the risk calculation unit to calculate a risk index for each health risk based on the manifest risk index and the potential risk index. FIG. 7 is a chart illustrating an exemplary functional material DB for a material selection unit to select a functional material(s) suitable for reducing a specific health risk. FIG. 8 is a view illustrating an exemplary display pattern of food product information on a terminal device.

As illustrated in FIG. 1, the food product information providing system 1 includes a plurality of terminal devices 10, a food product information presenting device 20, a food product production management device 30, and a food product delivery device 40.

The plurality of terminal devices 10 are terminal devices used by respective users, and are mutually communicably connected to the food product information presenting device 20 via a communication network N, such as the Internet. The plurality of terminal devices 10 are configured to be capable of outputting input information input from the respective users to the food product information presenting device 20, and receiving food product information described below from the food product information presenting device 20. It should be noted that the plurality of terminal devices 10 may be any information terminals that can perform communication, such as smartphones, tablet terminals, personal computers (PC), wearable information terminals, and Internet televisions.

The food product information presenting device 20 receives each information output from each of the plurality of terminal devices 10, and calculates a risk index for a health risk described below based on each information received. Then selects a functional material(s) that is/are effective in reducing the health risk with reference to the calculated risk index. Then, the food product information presenting device 20 outputs to the terminal device 10 food product information, such as a food product or recipe including the selected functional material(s). The detailed configuration and processing operation of the food product information presenting device 20 will be described below.

The food product production management device 30 and the food product delivery device 40 are connected to the food product information presenting device 20 via the communication network N.

The food product production management device 30 instructs a food product production unit (not illustrated) to produce a food product including a predetermined functional material(s) based on an instruction from the food product information presenting device 20.

The food product delivery device 40 instructs a delivery unit (not illustrated) to deliver a food product including a predetermined functional material(s) based on instructions from the food product information presenting device 20 and the food product production management device 30.

Next, as illustrated in FIG. 2, each terminal device 10 includes an information input unit 11, a message display unit 12, a food product information display unit 13, and a food product selection unit 14.

The food product information presenting device 20 includes a processor 100 and a storage unit 200.

The food product production management device 30 includes a food product production management unit 31.

The food product delivery device 40 includes a delivery instruction unit 41 and a delivery address DB 42.

It should be noted that each of the terminal device 10, the food product information presenting device 20, the food product production management device 30, and the food product delivery device 40 includes a processor, such as a CPU, and a storage device for storing various programs. Each of various functions is executed when a program is executed by each processor.

Hereinafter, an overview of the processing operation of the food product information providing system 1 will be described with reference to FIG. 3.

First, based on genetic information 111, biological information 112, and lifestyle information 113 input from the information input unit 11 of the terminal device 10, a risk calculation unit 120 of the food product information presenting device 20 calculates a potential risk index 121 and a manifest risk index 122 for each health risk, such as diabetes, dyslipidemia, or osteoporosis, and calculates a risk index (e.g., a weighted average risk index 123) for each health risk. Herein, the potential risk index is a numerical value indicating the risk level of getting a disease in the future, while the manifest risk index is a numerical value indicating the current risk level of having a disease. Next, if the risk index is greater than or equal to a first threshold and less than a second threshold, a material selection unit 130 selects one or more functional materials by prioritizing the effect for the relevant health risk or the price according to the proportion of potential risk index to manifest risk index in the risk index. Then, a food product information providing unit 150 of the food product information presenting device 20 outputs to the food product information display unit 13 of the terminal device 10 food product information on one or more food products including the one or more selected functional materials (information on a food product(s) including the functional material(s) or recipe information), and causes the food product information display unit 13 to display the information. On the one hand, if the risk index is greater than or equal to the second threshold, the user is determined to be in a disease state, and a message output unit 126 is instructed to output a predetermined message (e.g., a message recommending seeing a doctor) to the message display unit 12 of the terminal device 10. On the other hand, if the risk index is less than the first threshold, the user is determined to be in a healthy state, and the material selection unit 130 is instructed not to select functional materials. Herein, although the information input unit 11 is disposed in the terminal device 10 in the present embodiment, the present invention is not limited thereto. The information input unit 11 will be described in detail below.

Next, the configuration of the terminal device 10 will be described with reference to FIG. 2.

As illustrated in FIG. 2, the terminal device 10 includes the information input unit 11, the message display unit 12, the food product information display unit 13, and the food product selection unit 14.

The information input unit 11 is an input unit capable of receiving an input of the genetic information, biological information, and lifestyle information on a user. The information input unit 11 may be configured such that information such as those illustrated in FIGS. 4(a) to 4(c) can be input to a genetic information input unit 11a, a biological information input unit 11b, and a lifestyle information input unit 11c, respectively, for example.

Examples of the genetic information include the information illustrated in FIGS. 4(a) and 5. The genetic information as referred to herein is the results of genetic testing on an individual user received from a testing institution that provides a genetic testing service, and includes a risk value indicating the risk level as seen from a statistical mean value for each health risk. Examples of the biological information include the information illustrated in FIGS. 4(b) and 5, and include information on the results of a medical checkup. Examples of the lifestyle information include the information illustrated in FIGS. 4(c) and 5, and include lifestyle questionnaire information on the user. Herein, the same holds true for various types of information acquired by an information acquisition unit 110 described below.

In the present embodiment, although the information input unit 11 is disposed in the terminal device 10 and is configured to be capable of receiving an input of the genetic information, biological information, and lifestyle information on a user, the present invention is not limited thereto. For example, the information input unit 11 may be configured to be capable of receiving one or two of genetic information, biological information, and lifestyle information. Herein, as described above, the information input unit 11 may be disposed not only in the terminal device 10 but also in a terminal device different from the terminal device of the user, for example, a terminal device in a hospital or a genetic testing institution. In such a case, one or more of genetic information, biological information, and lifestyle information is input to the information acquisition unit 110 described below from the hospital or the genetic testing institution via a communication network N or a portable storage medium (for example, a USB). Alternatively, the information input unit 11 may he disposed in the food product information presenting device 20 and be configured to be capable of reading a document, which describes genetic information, biological information, or lifestyle information, sent from a user by mail. As a further alternative, the information input unit 11 may be constructed from a plurality of various terminal devices and be configured to receive various types of information concurrently or individually.

The message display unit 12 is configured to be capable of displaying a message output from the message output unit 126 of the food product information presenting device 20 described below. For example, the message output unit 126 outputs a message, which recommends seeing a doctor. Then, the message display unit 12 displays the message.

The food product information display unit 13 is configured to be capable of displaying food product information on one or more food products including the selected functional material(s) described below. The food product information represents, for example, information on a food product(s) including information on one or more functional materials selected based on information on a plurality of food products and the risk index of the user, or recipe information. The food product information display unit 13 is configured to be capable of displaying, for example, an image of the appearance of each food product, information such as the type(s) and mix proportion(s) of a functional material(s), price information, transition of the risk index, discount points, and selection icons partially forming the food product selection unit 14 described below in a pattern exemplarily illustrated in FIG. 8.

The food product selection unit 14 is configured to be capable of receiving an input of selection of a food product from a user who has referred to the food product information displayed on the food product information display unit 13, for example, the types of food products (the names of dishes or products) including the selected functional material(s). The food product selection unit 14 outputs the received instruction about the selected food product to a food product selection receiving unit 160 of the food product information presenting device 20 described below. The food product selection unit 14 is configured to allow the user to select a desired food product by designating a selection icon “Add to Cart” in the example illustrated in FIG. 8, for example.

The configuration of the food product information presenting device 20 will be described with reference to FIG. 2.

As illustrated in FIG. 2, the food product information presenting device 20 includes the processor 100 and the storage unit 200.

The processor 100 includes the information acquisition unit 110, the risk calculation unit 120, a risk index determination unit 125, the message output unit 126, the material selection unit 130, a price calculation unit 140, the food product information providing unit 150, and the food product selection receiving unit 160.

The information acquisition unit 110 is configured to be capable of acquiring the genetic information, biological information, and lifestyle information on a user received from the information input unit 11. In addition, the information acquisition unit 110 is configured to be capable of acquiring not only information from the terminal device 10 but also medical checkup information and genetic testing information on a user from a medical institution (together with a user ID), for example. Herein, examples of the information on the user include, but are not limited to, information on the results of genetic testing (genetic information), information on the results of a medical checkup (biological information), and information on the results of a lifestyle questionnaire (lifestyle information). The information acquisition unit 110 causes a user information DB 210 described below to store the various types of acquired information.

The risk calculation unit 120 selects one or more types of information from among the genetic information, biological information, and lifestyle information acquired by the information acquisition unit 110 according to each of a plurality of types of health risks, and calculates a potential risk index and a manifest risk index for each of the plurality of types of health risks based on the selected information. For example, the risk calculation unit 120 uses the genetic information, biological information, and lifestyle information on a user acquired by the information acquisition unit 110 and stored in the user information DB 210.

The risk calculation unit 120 calculates a manifest risk index and a potential risk index, and calculates a risk index based on the potential risk index and the manifest risk index.

The risk calculation unit 120 first calculates a manifest risk index and a potential risk index.

For calculating the potential risk index, the risk calculation unit 120 uses genetic information. The risk calculation unit 120 calculates the potential risk index using genetic information and biological information, for example. In such a case, the risk calculation unit 120 calculates the potential risk index by selecting one or more types of information suitable for the evaluation of the potential risk of each health risk from among a plurality of types of genetic information and selecting one or more types of information suitable for the evaluation of the potential risk of each health risk from among a plurality of types of biological information. Further, the risk calculation unit 120 calculates the potential risk index using lifestyle information in addition to the genetic information and biological information. In such a case, the risk calculation unit 120 calculates the potential risk index by selecting one or more types of information suitable for the evaluation of the potential risk of each health risk from among a plurality of types of lifestyle information, in addition to the aforementioned selected information.

For calculating the manifest risk index, the risk calculation unit 120 uses biological information and does not use genetic information. The risk calculation unit 120 calculates the manifest risk index by selecting one or more types of biological information suitable for the evaluation of the manifest risk of each health risk from among a plurality of types of biological information

The risk calculation unit 120 calculates a risk index based on the potential risk index and the manifest risk index, for example. For example, the risk calculation unit 120 can calculate the risk index by performing an operation on the potential risk index and the manifest risk index, such as addition, calculation using a calculation formula with a weighting factor, averaging, or weighted averaging. When the risk calculation unit 120 calculates the risk index by determining the weighted average of the potential risk index and the manifest risk index, for example, the risk index can be calculated from the following formula: the risk index=the potential risk index×W+the manifest risk index×(1−W), where W is the weighting factor (of less than 1).

A specific process of the risk calculation unit 120 will be further described with reference to FIG. 5.

As illustrated in FIG. 5, a health risk DB 220 described below stores determination items that are effective in determining each health risk, such as diabetes, dyslipidemia, or osteoporosis (information suitable for evaluating each health risk), the weighting factors of the determination items, and the like in association with each other. As illustrated in FIG. 5, the determination items that are effective in determining each health risk (information suitable for evaluating each health risk) differ depending on the types of health risks.

The risk calculation unit 120 calculates a potential risk index and a manifest risk index for each health risk of each user with reference to the health risk DB 220 described below. For example, the potential risk index of diabetes can be obtained by determining the weighted average of (1) a diabetes risk value obtained by genetic testing, (2) risk values obtained by normalizing the blood pressure and BMI (Body Mass Index), which are biological information, using their respective reference values, and (3) risk values obtained by normalizing an eating habit (an estimated weekly caloric intake), a smoking habit, a drinking habit, and a family history of diabetes, which are lifestyle information, using their respective reference values. Meanwhile, the manifest risk index of diabetes is obtained by determining the weighted average of risk values obtained by normalizing the blood glucose level and hemoglobin A1c (HbA1c), which are biological information, using their respective reference values.

The potential risk index of dyslipidemia can be obtained by, for example, determining the weighted average of (1) a dyslipidemia risk value obtained by genetic testing, (2) a risk value obtained by normalizing BMI, which is biological information, using its reference value, and (3) a risk value obtained by normalizing an eating habit (an estimated weekly caloric intake) and an exercise habit, which are lifestyle information, using their respective reference values. Meanwhile, the manifest risk index of dyslipidemia can be obtained by determining the weighted average of risk values obtained by normalizing LDL cholesterol, HDL cholesterol, and neutral fat, which are biological information, using their respective reference values.

The potential risk index of osteoporosis can be obtained by, for example, determining the weighted average of (1) an osteoporosis risk value obtained by genetic testing, (2) a risk value obtained by normalizing a bone formation marker and a bone resorption marker, which are biological information, using their respective reference values, and (3) a risk value obtained by normalizing an eating habit (an estimated weekly calcium intake), a smoking habit, a drinking habit, an exercise habit, and a sunlight exposure time, which are lifestyle information, using their respective reference values. Meanwhile, the manifest risk index of osteoporosis is a risk value obtained by normalizing bone density, which is biological information, using its reference value. It should be noted that the eating habit (an estimated caloric intake and an estimated calcium intake) may be estimated from photographs of the meals for the past week provided by the user, purchase history, or questionnaire. In addition, the smaller the absolute value of each of HDL cholesterol, bone formation marker, calcium intake, exercise frequency of an exercise habit, sunlight exposure time, and bone density, the higher the risk value.

It should be noted that the determination items for the potential risk index and the manifest risk index of each health risk are not limited to those described above, but for the determination of each health risk, at least one determination item is required for each of the potential risk index and the manifest risk index. The determination items of biological information that serve as the bases for calculation of the manifest risk index are preferably any of the determination items for diseases exemplarily listed in the guidelines of the Japan Society of Ningen Dock, the Japan Osteoporosis Society, the Japan Atherosclerosis Society, or the Japan Diabetes Society, for example. Meanwhile, the determination items for the potential risk index include genetic information and can also include some types of biological information and lifestyle information that indicate potential health risks.

The risk index determination unit 125 is configured to be capable of determining if the risk index calculated by the risk calculation unit 120 is greater than or equal to a predetermined first threshold. In addition, the risk index determination unit 125 is configured to be capable of determining if the risk index calculated by the risk calculation unit 120 is less than a predetermined second threshold. Further, the risk index determination unit 125 is configured to be capable of determining if the risk index calculated by the risk calculation unit 120 is greater than or equal to the predetermined first threshold and less than the predetermined second threshold.

The risk index determination unit 125 determines if the user is in a healthy state, a disease state, or a presymptomatic disease state based on the risk index.

The first threshold is a value set as a threshold for the healthy state and the presymptomatic disease state. The second threshold is a value set as a threshold for the presymptomatic disease state and the disease state.

In the present embodiment, if the risk index is less than the first threshold, the risk index determination unit 125 determines that the user is in a healthy state, and instructs the material selection unit 130 described below not to select functional materials. Specifically, the risk index determination unit 125 is configured to, if all of the risk indices for a plurality of health risks are less than the first threshold, instruct the material selection unit 130 not to select functional materials, and, if only some of the risk indices for the plurality of health risks are less than the first threshold (that is, if there is any other health risk having a risk index greater than or equal to the first threshold), instruct the material selection unit 130 to select a functional material(s) that is/are effective in reducing the health risk.

In addition, in the present embodiment, the risk index determination unit 125 is configured to, if the risk index is greater than or equal to the second threshold, determine that the user is in a disease state, and instruct the message output unit 126 described below to output a predetermined message (e.g., a message recommending seeing a doctor) to the message display unit 12 of the terminal device 10. Specifically, the risk index determination unit 125 is configured to, if all or some of the risk indices for a plurality of health risks are greater than or equal to the second threshold, instruct the message output unit 126 described below to output a predetermined message a message recommending seeing a doctor) to the terminal device 10.

Further, in the present embodiment, the risk index determination unit 125 is configured to, if the risk index is greater than or equal to the first threshold and less than the second threshold, determine that the user is in a presymptomatic disease state, and instruct the material selection unit 130 described below to select a functional material(s). Specifically, the risk index determination unit 125 instructs the material selection unit 130 to select a functional material(s) if all or some of the risk indices for a plurality of health risks are greater than or equal to the first threshold and less than the second threshold.

Specifically, in the example illustrated in FIG. 6, when the first threshold for the risk index is set to 2.0, the risk index determination unit 125 instructs the material selection unit 130 to refer to a functional material DB 230 described below for diabetes and dyslipidemia and to select a functional material(s) corresponding to diabetes and dyslipidemia.

If the risk index calculated by the risk calculation unit 120 is greater than or equal to the second threshold, the message output unit 126 outputs a predetermined message (e.g., a message recommending seeing a doctor). Specifically, if the risk index determination unit 125 determines that the risk index is greater than or equal to the second threshold, the message output unit 126 outputs a predetermined message (e.g., a message recommending seeing a doctor) to the message display unit 12 based on an instruction output from the risk index determination unit 125.

The material selection unit 130 selects one or more functional materials based on the risk index calculated by the risk calculation unit 120 for each of the plurality of types of health risks.

Based on the risk index calculated by the risk calculation unit 120, the material selection unit 130 extracts one or more specific health risks from the plurality of health risks, and selects a functional material(s) suitable for reducing the extracted one or more specific health risks.

Specifically, as described above, in the example illustrated in FIG. 6, when the first threshold for the risk index is set to 2.0, the material selection unit 130 extracts diabetes and dyslipidemia as specific health risks, and issues an instruction to select a functional material(s) corresponding to diabetes and dyslipidemia with reference to the functional material DB 230 described below.

The material selection unit 130 selects a functional material(s) according to any of (1) to (3) as follows:

(1) preferentially selecting a functional material that is relatively highly effective in reducing a specific health risk,

(2) preferentially selecting a functional material from a price perspective, and

(3) preferentially selecting a functional material that is effective in reducing a plurality of specific health risks.

Alternatively, the material selection unit 130 may be configured to select a functional material(s) based on a priority index set by a user using a priority index setting unit (not illustrated) included in the terminal device 10.

As described above, the material selection unit 130 selects a functional material(s) if the risk index calculated by the risk calculation unit 120 is greater than or equal to the first threshold. In the present embodiment, if the risk index of a specific health risk is greater than or equal to the first threshold and less than the second threshold, the material selection unit 130 selects one or more functional materials corresponding to the health risk according to the proportion of potential risk index to manifest risk index in the aforementioned risk index.

As a specific example, assume that the risk calculation unit 120 has calculated a manifest risk index and a potential risk index, and calculated a risk index based on the formula of the weighted average: “the risk index=the potential risk index×W+the manifest risk index×(1−W), where W is the weighting factor (of less than 1),” for example. Then, if the risk index of a specific health risk is greater than or equal to the first threshold and also if (1)“the manifest risk index×(1−W)” is greater than or equal to “the potential risk index×W,” that is, if the proportion of the manifest risk index in the risk index is higher than or equal to that of the potential risk index, the material selection unit 130 may be configured to preferentially select a highly effective functional material(s) because the user in a presymptomatic disease state is closer to a disease state, and if (2) “the manifest risk index×(1−W)” is less than “the potential risk index×W,” that is, if the proportion of the manifest risk index in the risk index is lower than that of the potential risk index, the material selection unit 130 may be configured to preferentially select an inexpensive functional material(s) and prompt the user to take in the selected functional material(s) for a long term because there is an ample time. It should be noted that the weighting factor W may be determined based on, for example, the reliability of the genetic information, biological information, and lifestyle information for each health risk that serve as the bases for calculating the potential risk index and the manifest risk index, or may be determined in other ways.

In addition, the material selection unit 130 determines the compatibility between the flavor of a food product to be presented to the user based on the preference information of the user stored in the user information DB 210 and the flavor of one or more functional materials selected by the material selection unit 130, and, if there is any functional material determined to have a flavor incompatible with the flavor of the food product to be presented, excludes such functional material and selects one or more functional materials again. As used herein, the term “flavor” represents a concept including taste and aroma, for example. Thus, in the determination of the compatibility between each functional material and each food product, the compatibility may be determined based on any of only taste, only aroma, and the whole flavor as an index. The preference information of the user is the information specifically related to the user's preference of the taste of foods, and includes, for example, information such that the user likes a “soy sauce taste ” and “pork taste” and does not like a “spicy” taste very much. Such preference information is estimated based on the purchase history information of the user, the results of a questionnaire included in the lifestyle information, and information on images of the meals of the user for the past week that are stored in the user information DB 210.

The material selection unit 130, based on the flavor information included in the food product information on a food product to be presented to the user based on the preference information of the user, and information on the flavor of each functional material included in the functional material DB described below (e.g., information on the compatibility with a specific flavor, information on the compatibility with a food product in a specific field, and information on the compatibility with a specific seasoning), evaluates the compatibility between the flavor of the food product to be presented and that of the functional material. Then, if there is any functional material determined to have a flavor incompatible with the flavor of the food product to be presented, the material selection unit 130 excludes such functional material, and selects one or more functional materials again. The re-selection may be performed either by excluding the functional material determined to have an incompatible flavor and selecting a functional material(s) from among all of the functional materials again, or by selecting only a material alternative to the excluded functional material (by replacing the excluded functional material with a new one).

A specific process of the material selection unit 130 will be further described with reference to FIG. 7.

In an exemplary list of the functional materials stored in the functional material DB 230 illustrated in FIG. 7, “Excellent” indicates a material that is effective in reducing a manifest health risk, and “Good” indicates a material capable of reducing a potential health risk.

An exemplary list of the functional materials illustrated in FIG. 7 will be further described below. As the functional materials corresponding to the health risk of diabetes, for example, indigestible dextrin, salacinol, partially hydrolyzed guar gum, and corosolic acid (derived from banaba leaves) are registered. Among them, corosolic acid is assigned “Excellent” as it is expected to be more effective in reducing the manifest health risk of diabetes than the other materials. In addition, as the functional materials corresponding to the health risk of dyslipidemia, for example, indigestible dextrin, EPA.DHA, and monoglucosyl hesperidin are registered. EPA.DHA and monoglucosyl hesperidin are assigned “Excellent” as they are expected to be more effective in reducing the manifest health risk of dyslipidemia than indigestible dextrin. Further, as the functional materials corresponding to the health risk of osteoporosis, for example, β-cryptoxanthin and soy isoflavone are registered. Among them, soy isoflavone is assigned “Excellent” as it is expected to be more effective in reducing the manifest health risk of osteoporosis than β-cryptoxanthin.

FIG. 7 also illustrates that indigestible dextrin is effective in reducing the potential risks of diabetes and dyslipidemia, and the material selection unit 130 is also capable of preferentially selecting a material that covers all of the plurality of identified health risks.

In the example of the user illustrated in FIGS. 5 and 6, when the first threshold is set to 2.0, the health risks of diabetes and dyslipidemia each have a risk index greater than or equal to the first threshold. Thus, functional materials corresponding to the health risks of diabetes and dyslipidemia are selected. In addition, since each of the health risks has a higher proportion of a potential risk index than that of a manifest risk index (case 1), functional materials are selected by prioritizing the price over the effect.

First, corosolic acid (price “3”) is the most inexpensive of all the functional materials that are effective in improving the health risk of diabetes, and indigestible dextrin (price “4”) is the most inexpensive of all the functional materials that are effective in improving the health risk of dyslipidemia. Therefore, a combination of corosolic acid and indigestible dextrin is selected as a first candidate. In such a case, the price of the functional materials as the first candidate is the total price “7” of corosolic acid and indigestible dextrin.

Next, a functional material that covers both the health risks of diabetes and dyslipidemia is searched for, and then, indigestible dextrin is selected as a second candidate. In such a case, one functional material can address the health risks of diabetes and dyslipidemia. Thus, the price of the functional material as the second candidate is “4.”

Finally, the price of the functional materials as the first candidate is compared with that of the functional material as the second candidate. Then, the more inexpensive functional mated al as the second candidate, that is, indigestible dextrin is selected.

It should be noted that when the potential risk is dominant for diabetes, and the manifest risk is dominant for dyslipidemia (case 2), corosolic acid (price “3”), which is the most inexpensive, is selected for diabetes by prioritizing the price, and one of EPA.DHA and monoglucosyl hesperidin is selected for dyslipidemia by prioritizing the effect. Herein, since both EPA.DHA and monoglucosyl hesperidin have the same effects, monoglucosyl hesperidin (price “12”), which is more inexpensive, is selected. In such a case, the total price of the functional materials is “15.”

When the manifest risk is dominant for diabetes, and the potential risk is dominant for dyslipidemia (case 3), corosolic acid (price “3”), which is assigned “Excellent” and thus is effective in improving the manifest risk of diabetes, is selected, and indigestible dextrin (price “4”), which is the most inexpensive, is selected for dyslipidemia. In such a case, the price of the functional materials is the total price “7” of corosolic acid and indigestible dextrin.

When the manifest risk is dominant over the potential risk for each of diabetes and dyslipidemia (case 4), corosolic acid (price “3”), which is assigned “Excellent” and thus is effective in improving the manifest risk of diabetes, is selected, and one of EPA.DHA and monoglucosyl hesperidin is selected for dyslipidemia by prioritizing the effect. Herein, since both EPA.DHA and monoglucosyl hesperidin have the same effects, monoglucosyl hesperidin (price “12”), which is more inexpensive, is selected. In such a case, the total price of the functional materials is “15.” Corosolic acid disclosed in FIG. 7 is effective in improving the manifest risk of diabetes, and is more inexpensive than the other materials (indigestible dextrin, salacinol, and partially hydrolyzed guar gum). Thus, the functional materials selected in the aforementioned case 4 are the same as those in the aforementioned case 2.

As described above, when the compatibility between the flavor taste) of a food product to be present to a user based on the preference information or purchase history information of the user, for example, and the flavor (e.g., taste) of the selected functional material is low, the material selection unit 130 may exclude such functional material and select a functional material(s) again.

Herein, the food product of the present embodiment contains, for example, a food ingredient and an additive material. Examples of food products include instant noodles in a container. Herein, a functional material(s) may be provided by being sealed in a dedicated bag, for example, but the present invention is not limited thereto. For example, a functional material(s) may be directly added to the food ingredient (for example, the portion of instant noodles)mixed in the additive material sealed in a separate bag, or further, mixed in a soup base sealed in a separate bag. In such a case, the material selection unit 130 is configured to be capable of proposing to increase or reduce the amount of the functional material(s) to be contained in the additive material. For example, the material selection unit 130 is configured to be capable of reducing the content of a specific functional material to be contained in the additive material. For example, when a food product is instant noodles in a container, the food product includes a noodle portion, which is the food ingredient, and an additive material sealed in a separate bag, for example. The material selection unit 130 is configured to be capable of issuing an instruction to increase or reduce the amount of a functional material(s) to be contained in the additive material and seal them in a bag, for example. Alternatively, when the additive material is sealed in a cup while being added to the noodle portion (i.e., not separately from the noodle portion), for example, the additive material with an increased or reduced amount of a predetermined functional material mixed therein is added to the noodle portion in the production stage. Further, when the additive material is not sealed in a separate bag but is contained in the soup base, the additive material with an increased or reduced amount of a predetermined functional material mixed therein is mixed in the soup base in the production stage.

The price calculation unit 140 is configured to be capable of calculating the food product price of one or more food products. The price calculation unit 140 is also configured to be capable of calculating discount points based on the frequency the user provides his/her health information (biological information or lifestyle information) and on the degree of improvement in the risk index. If the biological information (e.g., the results of a medical checkup) and lifestyle information (e.g., answers to a questionnaire) have been submitted frequently and the risk index has been decreased, discount points are added. Therefore, the price calculation unit 140 is configured to be capable of allowing a food product including a functional material(s) to be purchased at low price. The discount points are displayed on a purchase screen of the food product information display unit 13 of the terminal device 10 of the user so that the user can use the discount points each time he/she makes a purchase. In addition, the food product price may be set to automatically change according to the value of the discount points.

The food product information providing unit 150 provides food product information on one or more food products including one or more functional materials selected by the material selection unit 130, and one or more pieces of food product price information output from the price calculation unit 140. The food product information providing unit 150 is configured to be capable of outputting to the food product information display unit 13 of the terminal device 10 food product information including information on the selected one or more functional materials, information on one or more food products, and price information on the one or more food products. The food product information providing unit 150 selects one or more food products from among the food products stored in a food product DB 240 described below based on the preference information of the user stored in the user information DB 210 described below. Then, the food product information providing unit 150 outputs to the food product information display unit 13 of the terminal device 10 the food product information and food product price information on the food product(s) including the functional material(s). Herein, the food product information can include not only information on the functional materials and food products but also recipe information, for example.

The food product information providing unit 150 outputs to the food product information display unit 13 the food product information and price information on one or more food products including one or more functional materials, and causes the food product information display unit 13 to display the information on the one or more food products and the information on the one or more functional materials, for example.

For example, as illustrated in FIG. 8, the food product information providing unit 150 causes the food product information display unit 13 to display an image of the appearance of each of food products F1 to F5 including a functional material(s) based on the preference information of the user, and display the price information on the food product (e.g., F1) selected by the user as well as information on the type(s) and mix proportion(s) of the functional material(s). Herein, the type(s) and mix proportion(s) of the functional material(s) of the designated food product may be displayed in a pop-up by designating the image of the food product using a mouse, for example.

In addition, the food product information providing unit 150 may refer to the user information DB and cause the food product information display unit 13 of the terminal device 10 to display information on the risk index and its transition graph.

The food product information providing unit 150 may also be configured to be capable of outputting information for displaying a plurality of ordinary food products and causing the food product information display unit 13 of the terminal device 10 to display the plurality of such food products and also display one or more functional materials selected by the material selection unit 130 so as to allow the user to select a combination of an ordinary food product and a functional material(s) via the food product selection unit 14.

The food product selection receiving unit 160 receives an instruction about a selected food product from the user. Specifically, the food product selection receiving unit 160 receives selection information from the food product selection unit 14 of the terminal device 10.

Next, the storage unit 200 includes the user information DB 210, the health risk DB 220, the functional material DB 230, and the food product DB 240.

The user information DB 210 stores, for each user, personal information, such as a user ID and address, genetic information (e.g., the results of genetic testing) received in the past, biological information (e.g., the results of a medical checkup), lifestyle information (e.g., the results of a lifestyle questionnaire from the user), and the preference information of the user, for example. The user information DB 210 also stores information on the risk index calculated by the risk calculation unit 120 together with date and time information, for example.

The health risk DB 220 stores, as illustrated in FIG. 5, for example, health risk information on diabetes, dyslipidemia, and osteoporosis, determination items that are effective in calculating the risk index of each health risk (information suitable for evaluating each health risk), the weighting factors of the determination items, and the like in association with each other. As illustrated in FIG. 5, the determination items that are effective in determining each health risk (information suitable for evaluating each health risk) differ depending on the types of health risks.

The functional material DB 230 stores information, such as the prices of a plurality of functional materials, and information, such as the effectiveness of the functional materials for each health risk, in association with each other. The functional material DB 230 is configured to further store information on the compatibility between each functional material and each food product. For example, the functional material DB 230 stores, as information on the compatibility in terms of flavor, information on the compatibility with a specific flavor, information on the compatibility with a food product in a specific field, and information on the compatibility with a specific seasoning.

The food product DB 240 stores information on a plurality of food products. Examples of information on each food product stored in the food product DB 240 include information such as an image, composition, energy, allergy, and flavor (taste and aroma).

Next, the configuration of the food product delivery device 40 will be described with reference to FIG. 2.

As illustrated in FIG. 2, the food product delivery device 40 includes the delivery instruction unit 41 and the delivery address DB 42.

The delivery instruction unit 41 receives from the aforementioned food product selection receiving unit 160 food product information on a food product including a functional material(s) as well as information including the information for identifying at least the user. Then, the delivery instruction unit 41 acquires from the delivery address DB 42 delivery address information corresponding to the user ID, for example. Next, the delivery instruction unit 41 outputs to a delivery unit (not illustrated) the food product information and delivery address information as well as an instruction to deliver the food product. It should be noted that when a food product is produced after a user has purchased it, the delivery instruction unit 41 receives from the food product production management unit 31 of the food product production management device 30 information about the completion of production indicating that the production of the food product including a functional material(s) is complete.

Next, the configuration of the food product production management device 30 will be described with reference to FIG. 2.

As illustrated in FIG. 2, the food product production management device 30 includes the food product production management unit 31.

When a food product is produced after a user has purchased it, the food product production management unit 31 receives from the aforementioned food product selection receiving unit 160 food product information(including recipe information) on a food product including a functional material(s) selected by the user, and outputs the food product information to the food product production unit (not illustrated) to instruct it to produce the food product. When the production of the food product including the functional material(s) is complete, the food product production management unit 31 outputs information about the completion of the production to the delivery instruction unit 41 described below.

The delivery address DB 42 stores the user ID information and deliver address information (e.g., postal address or information on the workplace) association with each other.

Herein, the present invention includes not only the aforementioned food product information providing system and device, but also the following food product information providing method and program. An exemplary food product information providing method and an exemplary food product information providing program will be described below.

An exemplary food product information providing method is a method for proposing a food product including one or more functional materials, using a food product information providing device, for example, and includes an acquisition step of acquiring genetic information, biological information, and lifestyle information on a user, a risk calculation step of selecting one or more types of information from among the genetic information, biological information, and lifestyle information acquired in the acquisition step according to each of a plurality of types of health risks, and calculating a risk index for each of the plurality of types of health risks based on the selected information, a functional material selection step of selecting one or more functional materials based on the risk index for each of the plurality of types of health risks calculated in the risk calculation step, and a food product information providing step of providing food product information on one or more food products including the one or more functional materials selected in the functional material selection step.

An exemplary food product information providing program is a program for causing a food product information providing device to execute a food product information providing method for providing a food product including one or more functional materials, for example. The program causes the food product information providing device to execute the food product information providing method that includes an acquisition step of acquiring genetic information, biological information, and lifestyle information on a user, a risk calculation step of selecting one or more types of information from among the genetic information, biological information, and lifestyle information acquired in the acquisition step according to each of a plurality of types of health risks, and calculating a risk index for each of the plurality of types of health risks based on the selected information, a functional material selection step of selecting one or more functional materials based on the risk index for each of the plurality of types of health risks calculated in the risk calculation step, and a food product information providing step of providing food product information on one or more food products including the one or more functional materials selected in the functional material selection step.

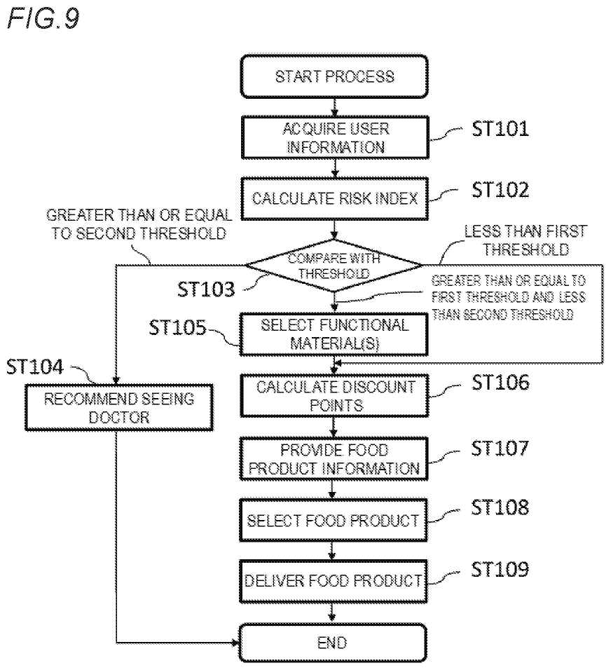

Next, the processing operation of the food product information providing system 1 will be described with reference to FIG. 9. FIG. 9 is a flowchart illustrating the processing operation of the food product information providing system of the present embodiment.

First, in step ST101, the information acquisition unit 110 acquires genetic information, biological information, and lifestyle information on a user, and stores the acquired information into the user information DB 210 in association with the user ID of the user.

Next, in step ST102, the risk calculation unit 120 calculates a risk index based. on the genetic information, biological information, and lifestyle information on the user stored in the user information DB 210. The risk calculation unit 120 stores the risk index in association with the user ID of the user and also in association with date and time information.

Next, in step ST103, the risk index determination unit 125 compares the calculated risk index with the first threshold and the second threshold. Then, if the calculated risk index is greater than or equal to the second threshold, the risk index determination unit 125 causes the message output unit 126 to output information, such as a message recommending seeing a doctor. Accordingly, the message display unit 12 of the terminal device 10 displays the message recommending seeing a doctor, for example, such that it can be viewed by the user. In this manner, when the user is determined to be in a disease state, the user is caused to view a message recommending seeing a doctor, for example.

Further, if the calculated risk index is less than the first threshold, the risk index determination unit 125 instructs the material selection unit 130 not to select functional materials. Accordingly, the process proceeds to step ST106. In this manner, when the user is determined to be in a healthy state, the material selection unit 130 is not caused to select functional materials.

Further, if the calculated risk index is greater than or equal to the first threshold and less than the second threshold, the risk index determination unit 125 instructs the material selection unit 130 to select a functional material(s). In this manner, when the user is determined to be in a so-called presymptomatic disease state, the material selection unit 130 is caused to select a functional material(s).

Next, in step ST105, the material selection unit 130 selects a plurality of functional materials that are effective in reducing a health risks) with a high risk index, for example. Herein, the material selection unit 130 selects a functional material that is highly effective in reducing a specific health risk, has a reasonable price, or is effective in reducing a plurality of specific health risks, for example, as a priority index. In addition, if the compatibility between the flavor of the functional material selected by the material selection unit 130 and the flavor of a food product to be presented to the user based on the preference information of the user is low, the material selection unit 130 selects one or more functional materials again. Herein, the term “flavor” represents a concept including taste and aroma, for example. Thus, in the determination of the compatibility between each functional material and each food product, the compatibility may be determined based on any of only taste, only aroma, and the whole flavor as an index.

Next, the price calculation unit 140 calculates discount points based on the frequency the user provides his/her health information (biological information or lifestyle information) and on the degree of improvement in the risk index, and outputs the value of the discount points to the food product information providing unit 150. For example, when the user provides his/her health information to the food product information presenting device 20, the user will earn one point added to his/her discount points, and when the risk index becomes lower, the user will further earn some more points added to his/her discount points according to the degree of improvement. The discount points can be consumed to reduce the food product price in response to a request from the user. In addition, the food product price may be set to automatically change according to the value of the discount points.

Next, in step ST107, the food product information providing unit 150 outputs to the food product information display unit 13 of the terminal device 10 food product information on a plurality of food products including the aforementioned selected functional material(s). Accordingly, the user is able to view a plurality of pieces of food product information including the functional material(s) information on an image of each food product, information on the functional material(s), price information, and discount points) on the terminal device 10.

Next, in step ST108, the food product selection unit 14 of the terminal device 10 receives an input of selection of a food product from the user, and outputs to the food product selection receiving unit 160 of the food product information presenting device 20 selection information including information that identifies the food product selected by the user. Then, the food product selection receiving unit 160 receives the selection information, and outputs to the delivery instruction unit 41 of the food product delivery device 40 food product information on a food product identified from the selection information as well as the user ID information that identifies the user. It should be noted that when the food product is produced after the user has purchased it, the food product selection receiving unit 160 also outputs to the food product production management unit 31 of the food product production management device 30 the selection information including information that identities the food product including the functional material(s) selected by the user.

Next, in step ST109, the delivery instruction unit 41 of the food product delivery device 40, upon receiving the food product information and the user ID information from the food product selection receiving unit 160, outputs the food product information and the user delivery address information stored in the delivery address DB to the delivery unit (not illustrated) and instructs it to deliver the food product. Then, the processing operation of the food product information providing system 1 terminates.

According to the food product information providing system 1 of the present embodiment, the following advantageous effects are achieved.

According to the present embodiment, a user can easily and effectively take in functional materials that are effective in reducing his/her health risks by eating a food, such as a processed food product that he/she purchases on a daily basis.

According to the present embodiment, a user can unconsciously take in a food product including effective functional materials according to his/her potential health risk and manifest health risk.

It should be noted that the present invention is not limited to the aforementioned embodiments. Thus, the present invention includes any variations and modifications that are made within the spirit and scope of the present invention.

The present application is based on Japanese Patent Application No. 2018-108125 filed on Jun. 5, 2018, the entire content of which is hereby incorporated by reference into this application.

### REFERENCE SIGNS

1 Food product information providing system
10 Terminal device
11 Information input unit
12 Message display unit
13 Food product information display unit
14 Food product selection unit
20 Food product information presenting device
30 Food product production management device
31 Food product production management unit
40 Food product delivery device
41 Delivery instruction unit
42 Delivery address DB
100 Processor
110 Information acquisition unit
120 Risk calculation unit
125 Risk index determination unit
126 Message output unit
130 Material selection unit
140 Price calculation unit
150 Food product information providing unit
160 Food product selection receiving unit
200 Storage unit
210 User information DB
220 Health risk DB
230 Functional material DB
240 Food product DB

1. (canceled)

2. A food product information providing device comprising:
an information acquisition unit that acquires genetic information, biological information, and lifestyle information on a user;
a risk calculation unit that selects one or more types of information from among the genetic information, the biological information, and the lifestyle information acquired by the information acquisition unit according to each of a plurality of types of health risks, and calculates a risk index for each of the plurality of types of health risks based on the selected information;
a material selection unit that selects one or more functional materials based on the risk index for each of the plurality of types of health risks calculated by the risk calculation unit; and
a food product information providing unit that provides food product information on one or more food products including the one or more functional materials selected by the material selection unit.

3. The food product information providing device according to claim 2, wherein the risk calculation unit calculates a manifest risk index and a potential risk index, and calculates the risk index based on the potential risk index and the manifest risk index.

4. The food product information providing device according to claim 3, wherein the risk calculation unit uses at least the genetic information to calculate the potential risk index, and uses at least the biological information to calculate the manifest risk index.

5. The food product information providing device according to claim 3, wherein the material selection unit extracts from the plurality of types of health risks one or more health risks each having a risk index greater than or equal to a first threshold as one or more specific health risks, and selects a functional material suitable for reducing the extracted one or more specific health risks based on the potential risk index and the manifest risk index.

6. The food product information providing device according to claim 5, wherein the material selection unit selects the functional material according to any of (1) to (3) as follows:
(1) preferentially selecting a functional material that is relatively highly effective in reducing a specific health risk,
(2) preferentially selecting a functional material from a price perspective, and
(3) preferentially selecting a functional material that is effective in reducing a plurality of specific health risks.

7. The food product information providing device according to claim 5,
wherein the risk calculation unit calculates the manifest risk index and the potential risk index, and calculates as the risk index a weighted average value of the manifest risk index and the potential risk index, and
wherein the material selection unit preferentially selects the following as a functional material suitable for reducing the specific health risk:
(1) a highly effective functional material if a proportion of the manifest risk index in the risk index is higher than or equal to a proportion of the potential risk index, or
(2) an inexpensive functional material if the proportion of the manifest risk index in the risk index is lower than the proportion of the potential risk index.

8. The food product information providing device according to claim 5, further comprising a message output unit that outputs a predetermined message if the risk index is greater than or equal to a second threshold.

9. The food product information providing device according to claim 2, wherein the material selection unit determines a compatibility between a flavor of a food product selected based on preference information of the user and a flavor of the one or more functional materials selected by the material selection unit, and if there is any functional material determined to have a flavor with a low compatibility with the flavor of the selected food product, excludes the functional material determined to have the flavor with the low compatibility and then selects one or more functional materials again.

10. The food product information providing device according to claim 2,
wherein the food product includes a food ingredient and an additive material, and
wherein the material selection unit is configured to be capable of proposing to increase or reduce an amount of the functional material to be contained in the additive material.

11. The food product information providing device according to claim 2, further comprising a price calculation unit that calculates a food product price of the one or more food products presented by the food product information providing unit, and
wherein the price calculation unit is configured to be capable of calculating a discount point based on a frequency the user provides his/her health information and on a degree of improvement in the risk index, and changing the food product price according to the discount point.

12. A food product information providing method for proposing a food product including one or more functional materials using a food product information providing device, comprising:
an acquisition step of acquiring genetic information, biological information, and lifestyle information on a user;
a risk calculation step of selecting one or more types of information from among the genetic information, the biological information, and the lifestyle information acquired in the acquisition step according to each of a plurality of types of health risks, and calculating a risk index for each of the plurality of types of health risks based on the selected information;
a functional material selection step of selecting one or more functional materials based on the risk index for each of the plurality of types of health risks calculated in the risk calculation step; and
a food product information providing step of providing food product information on one or more food products including the one or more functional materials selected in the functional material selection step.

13. A non-transitory computer-readable recording medium storing a food product information providing program for causing a food product information providing device to execute a food product information providing method for proposing a food product including one or more functional materials, the method including:
an acquisition step of acquiring genetic information, biological information, and lifestyle information on a user;
a risk calculation step of selecting one or more types of information from among the genetic information, the biological information, and the lifestyle information acquired in the acquisition step according to each of a plurality of types of health risks, and calculating a risk index for each of the plurality of types of health risks based on the selected information;
a functional material selection step of selecting one or more functional materials based on the risk index for each of the plurality of types of health risks calculated in the risk calculation step; and
a food product information providing step of providing food product information on one or more food products including the one or more functional materials selected in the functional material selection step.
